(19) United States
(12) Patent Application Publication
(10) Pub. No.: US 2009/0253974 A1
(43) Pub. Date: Oct. 8, 2009

(54) Use of tools, mapping systems, catheters, electrodes or any devices targeting any autonomic nerve(s) structure(s) in the human heart for the diagnostic, treatment and/or prevention of the recurrence of cardiac arrhythmias.

(76) Inventor: Marc Mounir Rahme, Ottawa (CA)

Correspondence Address: Marc M. Rahme, 107 Flamborough Way, Kanate, ON, K2K 2E9, CA

(21) Appl. No.: 12/099,225

(22) Filed: Apr. 8, 2008

(51) Int. Cl. A61B 5/04; A61B 18/14; A61M 31/00

(52) U.S. Cl. 600/372; 606/41; 604/500

(57) ABSTRACT

Treatment of atrial fibrillation, targeting the triggering autonomic nerve structures have been shown high efficacy for converting to a sinus rhythm state. However, the efficacy is limited to a short time (<3 months) with a high degree of recurrence of atrial arrhythmias. The recurrence rate was directly correlated to the time of follow-up which would support the role of the autonomic nerve regenerative (or re-establishment) activities for the incidence of the recurrence of atrial arrhythmias (ex. persistent atrial fibrillation). Furthermore, the recurrence degree of atrial fibrillation has not been shown any statistically significant difference between any of the currently used methods within any targeted area. Therefore, the long term inefficacy matter could be related either to the existing technologies or to their uses. The autonomic nerve’ rotors which had triggered first the occurrence of atrial fibrillation would be regenerated its primary activities over time and therefore, triggering again their recurrence.

### V. BACKGROUND OF THE INVENTION

The treatments of atrial fibrillation are not yet proved effective and stable for long term despite a diversity of the available technologies. The rate of the recurrence is high and has not shown any statistically significant difference between different groups of patients treated by any existing methods (either devices, drugs or both). Therefore, the hypothesis of a recurrence rate (high and not statistically different between all treated groups of patients) would be related to a common primer electro-physiological cause ‘nest’ such as the vagal nerve. It is more scientifically reliable to re-investigate the primary causes, rather than the difference in technologies, devices, drugs and/or anatomical targeted areas. Therefore, these electrophysiological cause(s) could be related to regenerative phenomena of the autonomic nerve structure which may be correlated directly or indirectly to the premium causes of the initiation/occurrence of those cardiac arrhythmias. It would be evidence to investigate this hypothesis by studying either or not the recurrence rate would be correlated to the nerve regenerative matter between different drugs and technologies.

The most recent studies from all over the world have been added clinical support and values to my previous fundamental discovery on the primordial role of the autonomic nervous system on the initiation, maintenance and persistence of atrial fibrillation. However, the recurrence, a major limitation of my previous patent, was not related to the quality of scientific results and interpretations, rather than to the duration of the studies which were performed in acute models. Therefore, the recurrence matter was not clearly showed or interpreted despite some theoretical attempts and scientific interpretations. Currently, several clinical studies which were conducted by well known world wide clinical centers are supporting the implications of the autonomic nervous system in the occurrence of atrial fibrillation with a high degree of recurrence rate. Furthermore, it is obvious that a nerve tissue could regenerate its electrophysiological conductive abilities following a partial or an incomplete catheter ablation procedure which could reverse as well the effect of the catheter ablation procedure.

Therefore, a hypothesis based on using an appropriate technology for ablating a nerve tissue and preventing permanently its conductive regenerative ability would be scientifically relevant. Furthermore, a method of ablating a nerve tissue by using the existing technology without ensuring the complete and irreversibility of nerve re-conduction thereby could lead to nerve regeneration of the nerve structure (ansa, Ganglionated Plexi, nerve fibers, etc) which may lead as well to the recurrence of atrial arrhythmias, in particular atrial fibrillation, a major cardiac disease in our aging population.

The main purpose of the present invention is highlight new finding that emerged in the complications of the autonomic nervous system on the occurrence, persistence and recurrence of atrial arrhythmias, in particular the early recurrence of atrial fibrillation. Furthermore, the development of treatment methods for atrial fibrillation in order to prevent more complications and site effects (as listed below), the early- and/or late-recurrence would be the main priority of this invention. The outcomes results of the recurrence of atrial arrhythmias by using any of the existing treatment methods are not statistically significant difference (between the groups which were received the treatments—methods of treatment; either catheter ablation or antiarrhythmic drugs; catheter ablation approaches/methods, etc). Furthermore, the correlation between the recurrence rate of AF and the time following the treatment of atrial fibrillation would support as well my scientific interpretations and observations on the implications over time of the autonomic nerve regenerative (or re-establishment) activities for the incidence of recurrence of atrial arrhythmias (more specifically following the treatment of the persistent atrial fibrillation). Therefore, the present invention is focusing on the outcomes recurrence of atrial fibrillation between the following methods:

1—Cox-Maze by use of bipolar radio frequency ablation.

### 2—Circumferential Pulmonary Vein Ablation (CPVA).

3—Linear ablation.
4—Segmental isolation. The recurrence was common despite that the 4 veins were isolated by using an open irrigated tip catheter and despite multiple procedures were applied as well.
5—Pulmonary vein isolation using a transvenous curvilinear cryoablation catheter.
6—Bi-directional electrical pulmonary vein isolation.
7—Ablation of the pulmonary veins antral isolations as determined by multi-detector computed tomography.

It is well known to our scientific arena that any hierarchic catheter ablating ‘method used without focusing on prior proven effectively’ ones coupled by a trustable mapping system (ex. Ensite System.) could be exposed to several sides effects such: decreasing of heart failure status (decrease systolic and diastolic functions, etc.), producing ischemic status, causing complications such as necrosis, coagulate necrosis, mitral regurgitation, embolic complications, neural dysfunction, recurrent laryngeal nerve damage, atrio-bronchial fistula, atrio-esophageal fistula, pericardial effusion, coronary lesions, cardiac tamponade, pulmonary vein stenosis, phrenic nerve injury, damage to collateral structures such as the esophagus, phrenic nerve and recurrent laryngeal nerve. For example, according to a recent clinical study, a patient had developed a left atrial-bronchial fistula following left atrial ablation, which required surgical repair.

In general and briefly, a hierarchic random method for catheter ablation used for the treatment of atrial fibrillation could lead as well to many majors' complications, side effects, inefficacy and early recurrence of cardiac arrhythmias. These complications could include and limited to the following: Electrophysiological changes such as loss of normal adaptation of atrial refractoriness to heart rate, contractile changes such as reduce atrial contractility, structural changes such as left atrium and left atrial appendage enlargement, decrease in cardiac output and histological changes.

In addition, many other factors could be promoted by the high recurrence rate of atrial fibrillation as well such as:

1—Prothrombotic changes (increased propensity for clot formation) such as atrial stasis and increases prothrombotic factors.
2—Enlarged atrial cells, severe myolysis, glycogen accumulation and reduction in connexin 40 expression
3—Increase the mortality rate, increase the persistence of stroke risk, increase the duration of the hospitalization and reduce the quality of life of the patients.

Several other factors could be related to the use of a hierarchic technology such as the risk of thrombosis' formation which was found directly correlated to the size of electrode and to the amount of delivered radiofrequency energy. The risks and sides effects related to some anatomical areas during catheter ablation's procedure could be resumed as risk of ablating the right superior pulmonary vein would be affecting the phrenic nerve, risk of ablating the right inferior pulmonary vein would be of damaging the vagi nerve and perforate the esophagus.

In conclusion, several advantages for the development of specific tools for targeting the autonomic nervous structure, improving the occurrence and preventing the recurrence issues of atrial fibrillation (ex. a neuro-cardio-stimulator). The ‘occurrence’ and the ‘recurrence’ of atrial fibrillation could be directly dependant to the same area (a specific area of nerve structure could lead to early activities for triggering atrial fibrillation and it could as well be reactivated after an ablation procedure to trigger again its recurrence by the regenerative activities matter). It could also be indirectly related to the imbalance or over activation of other branches of vagal nerve structures' activities. The vagi reach the heart into adjacent ganglia to the Heart Helium junction (Pulmonary vein Antrum, Vein Cava Antrum; =vagus is anatomically parallel to the pulmonary vein for example) (Pauza D., Armour A., Rahme M., etc.). It has been shown that isolating the PV Antrum in some patients could directly influence the vagal neural activities in a large area of the heart which was shown an effective approach to treat the occurrence, but not the recurrence, of atrial fibrillation. Therefore, developing a specific tool such as a neuro-cardiac-stimulator combined to a mapping system could lead to target directly the earliest activation within the nerve structure and thereby preserving other parts of the nerve which could preserve the same efficacy with a significantly decrease on the complications including and not limited to the early recurrence of those atrial arrhythmias. Furthermore, it could lead to much more discoveries for the treatment of atrial fibrillation and other major cardiac diseases such as sudden cardiac death. I strongly believe that a neuro-cardio-stimulator would be able to identify the nerve structure and their roles prior and during the occurrence of cardiac arrhythmias. It would be extensively used in order to prevent the regenerative activities as well of an originally targeted nerve for catheter ablation procedure. Furthermore, It could investigated as well many others scientifically and clinically relevant issues, such as the intrinsic autonomic activity within the smooth muscle sleeves of the heart myocardium, including the pulmonary veins areas, can trigger electrical activation which leads to atrial fibrillation; localizations of areas of inter-connections between both sympathetic and parasympathetic systems; understanding, resolving many electro-physiological-anatomical issues such as the balance of both systems within the same endocardial, myocardial and epicardial areas. Drs. Scherlag B J., Lin J., Zhou J., Song J., Zhang Y., Lazzara R., Patterson E., Po S S and Jackman W M have clearly stated that their fundamental study provides functional evidence for the interconnections between ganglionated plexi to modulate sinus and AV nodal function, supporting clinical evidence that interconnections within the intrinsic cardiac ANS are critical elements in identifying the targets for atrial fibrillation ablation. Drs. Packer team from Mayo Clinic, MN have disclosed as well the increasing rate of the recurrence of atrial fibrillation over time (beyond 18 months) and more specifically for the persistent atrial fibrillation (“Unlike Paroxysmal AF where recurrence plateaus from 12 to 24 months to 11%, recurrence rate rises in Permanent AF group from 14% to 20%”). Furthermore, they were able to questioning the concept of recurrence in persistent AF as a result from an additional substrate-mediated arrhythmia which was not be modifiable by the catheter ablation manner. This invention is suggesting and clarifying for the first time that those additional substrates would be related to the autonomic nervous system regenerative substrates such activities/tonicities/anatomies/restructuring/etc (re-activation, re-establishment, ionic and/or molecular changes, etc.).

### VI. BRIEF SUMMARY OF THE INVENTION

In brief, this invention is specifying new targets and arenas for the effective uses of catheter ablation methods and for the anti-arrhythmic drugs on treatment and prevention of the recurrence of atrial fibrillation.

The main purpose of the present invention is to highlight new finding that emerged in the implications of the autonomic nervous system regenerative activities on the early recurrence of atrial fibrillation.

The development of treatment methods for atrial fibrillation in order to prevent more complications and site effects (as listed in the Background section), the early- and/or late-recurrence would be the main priority of this invention. The outcomes results of the recurrence of atrial arrhythmias by using any of the existing treatment methods are not statistically significant difference (between the groups which were received the treatments—methods of treatment; either catheter ablation or antiarrhythmic drugs; catheter ablation approaches/methods). Furthermore, the correlation between the recurrence rate of AF and the time following the treatment of atrial fibrillation would support as well the scientific interpretations on the direct role over time of the autonomic nerve regenerative (or re-establishment) activities for the incidence of recurrence of atrial arrhythmias (more specifically following the treatment of the persistent atrial fibrillation).

The currently used treatments for the occurrence of atrial fibrillation would be more effective as long as it could prevent the recurrence of the targeted autonomic nerves' triggers to re-generate (/re-conduct) substrates. Thereby, the nerve regenerative activities caused by electrical reconditioning (/re-establishment) of the ablated (/targeted) areas could be the main cause for the inefficacity of the conventional catheter ablation’ methods used for treatment of atrial arrhythmias. Therefore, using tools, mapping systems, catheters, electrodes or neuro-devices for preventing the re-activation of neural abilities of areas targeted on the treatment of atrial fibrillation would significantly prevent the recurrence of atrial arrhythmias.

In addition, the scientific interpretations and conclusions would valid as well the matter that the existing’ methods used for ablating the nerve structure on the treatment of atrial fibrillation are encountered a major long term inefficacy such as high incidence of recurrence. Thus, it could be related either to their inability to terminate completely the arrhythmic activities of the said ‘nerve structure’, or to prevent the targeted nerve structures from re-establishing innervations over time such as regenerative and/or collateral’ neuron-arrhythmic activities.

### VII. BRIEF DESCRIPTION OF THE SEVERAL VIEWS OF THE DRAWING

None

### VIII. DETAILED DESCRIPTION OF THE INVENTION

### A. Words from the Inventor

“We are all together (Scientists, Physicians, devices and pharmaceutical companies) promising to offer innovative and effective approaches to a long term ‘permanent’ treatment for cardiac arrhythmias, in particular atrial fibrillation—Importance of structuring the autonomic nervous system, physiological rotors which triggering, monitoring, balancing and perpetuating those cardiac arrhythmias. In the treatment of a major cardiac disease, atrial fibrillation in particular, we are promising to use all available and up-to-date technologies such as mapping/targeting autonomic nervous structure by using local catheter ablation to prevent nerve regenerative activities for a long term ‘permanent’ treatment”. “In brief, the recurrence of atrial arrhythmias following treatment by using the conventional methods could be related to the nerve regenerative activities”. (Marc Mounir Rahme, PhD).

### B. United States Patent and Trade Office around the Globe

The present invention is related to many worldwide scientific studies following the publications of the U.S. Pat. No. 6,511,500 patent by the USPTO. Therefore, the atrial fibrillation (AF) patients from worldwide were able to receive an effective, accurate and up-to-date treatment. However, the '500 patent was based on acute studies and therefore it was limited to claim the AF recurrence matter.

The following are showing a few samples from the use of the '500 patent around the world:

1) A team of investigators for China (Dr. Chen SA., et al., in collaboration with Taiwan) had reported that the fixed complex fractionated atrial electrograms play an active role in atrial fibrillation perpetuation. However, the non-fixed complex fractionated electrograms may be related with the passive activation from the remote atrial fibrillation sources, not its driver(s). Furthermore, the complex fractionated atrial electrograms have been proposed to play a role in perpetuation of atrial fibrillation. It has been reported by several investigators from China (Drs Zhang S., Yang Y. et al., from Dalian, China) that pulmonary vein isolation attenuates the vagal modulation to the atria, thereby decreases the susceptibility to atrial fibrillation triggered by vagus. Furthermore, the pulmonary vein isolation releases the atrial remodeling, which maybe contributes to the vagal dennervation. Another team of investigator from Dalian, China had reported that decreased effective refractory period shortening to vagal stimuli after right upper pulmonary vein isolation indicates that it might result in the damage of the epicardia fat pad, Thereby attennate the vagal innervation to atrial. The decreased vulnerability window to atrial fibrillation by this isolation. Thus, it had indicated that attenuated vagal tone may contribute to the suppression of atrial fibrillation.
2) A Russian team of investigators (Dr. Pokushalov E., Turov A., Shugaev P, Artemenko S.) had reported that the ablation of ganglia is efficient approach in the treatment of AF. Vagal bradycardia intensity during RF ablation and degree of autonomic HRV modification after procedure are important predictors of the subsequent clinical effect.
3) The Euro Heart Survey is the first large observational study to provide a complete picture of AF triggers, autonomic trigger patterns, its management and outcome. An autonomic trigger pattern was frequently seen in paroxysmal AF patients. In contrast to the general opinion, we observed that vagal AF is not restricted to the young and healthy. The high percentage of inappropriate treatment suggests insufficient appreciation for autonomic triggers of AF. This may lead to aggravation of the arrhythmia
4) A team from Sweden (Drs. Blomstrom C M., et al) have used the video-assisted epicardial pulmonary vein (PV) and ganglionic plexi (GP) ablation off-pump as a new technique of atrial fibrillation (AF) ablation. They have report their initial experience including patients (pats) with permanent AF. Their results were as the following: PV isolation, vagal GP ablation and excision of LAA was successful in 28/30 (93%) pats. The pats were followed for 3,6,12 months after ablation. In 14 pats followed for 6 months and 3 pats for 12 months, 16 out of 17 (94%) are in sinus rhythm (SR), of whom only three are on antiarrhythmic drug treatment. All 4 pats followed for 3 months are in SR, 7 remains to be followed at 3 months and one pat is lost to follow-up. Complications included bleeding requiring thoracotomy in 2 pats and embolic stroke in one pat. They have concluded that video-assisted thoracoscopic epicardial PV isolation “off-pump” is feasible and well tolerated in AF patients. Our initial experience offers promising results for AF patients who cannot be cured by conventional AF catheter ablation, including those with permanent AF. The long term safety and efficacy of the method needs to be further evaluated, especially regarding necessity and efficacy of vagal denervation.

Another team from Sweden (Blomstrom C. et al.,) has studied the effects epicardial ganglionated plexi and pulmonary vein ablation on the treatment of atrial fibrillation. They have reported on a group of 30 patients (21 men and 9 women) that Video-assisted thoracoscopic epicardial PV isolation “off-pump” is feasible and well tolerated in atrial fibrillation patients. Our initial experience offers promising results for AF patients who cannot be cured by conventional AF catheter ablation, including those with permanent AF. The long term safety and efficacy of the method needs to be further evaluated, especially regarding necessity and efficacy of vagal denervation. Vagal ganglia sites were identified using high frequency stimulation (800 bpm, pulse width 9.9 msec) at specific predetermined sites around all pulmonary veins, by a decrease exceeding 50% of the ventricular rate during stimulation, and then ablated using radiofrequency energy. Vagal denervation was confirmed by repeating the GP stimulation after the radiofrequency application(s).

5) A team from Spain in collaboration with Syracuse, N.Y. (Dr. Atienza F., Jalife J., et al) results had demonstrated for the first time in humans that real-time DF mapping prior to ablation is a promising approach to aid in paroxysmal AF substrate ablation. Ablation at DFmax sites results in prolongation of the AFCL and termination of paroxysmal AF in a significant proportion of pts, confirming their role in the maintenance of AF.
6) A team from Seoul, Republic of Korea (Dr Park K., et al.) had also reported that the shortening of the effective refractory period of fast pathway were observed both after cryo- and radiofrequency ablation, without significant changes in autonomic activity. These results suggest that the electrotonic influence is the main mechanism for the changes of electrophysiologic properties of the fast pathway after cryoablation.
7) A team of investigators from, France (Bordeaux Drs. Jais P, Klein G. Haissaguerre P., et al.) had reported on the diagnostic approach to atrial tachycardia after ablating atrial fibrillation. This diagnostic approach is practical and straightforward in determining the origin of atrial tachycardia occurring after AF ablation. In their approach, the firing autonomic rotors might be targeted to achieve acceptable clinical outcomes.
8) A team of investigators from the University of Adelaide, Australia (Dr. Stiles MK. Et al.,) had reported that the sites of complex fractionated atrial electrograms (CFAEs) and dominant frequency (DF) have been proposed as critical regions maintaining atrial fibrillation (AF). The optimal duration of electrogram analysis required to accurately define these regions is not known. While longer electrogram recordings may not be practical, shorter periods may provide inaccurate representation. In conclusions in 10 patients with permanent atrial fibrillation selected for catheter ablation procedures, the length of electrogram segment analyzed is more important for DF analysis than CFE mean. Electrogram recordings as short as 2 seconds may be sufficient for CFE mean analysis, however more than 4 seconds are required for DF analysis to approximate results from longer recordings.
9) A team from Sao Paulo, Brazil (Dr Scanavacca M. et al.,) had studied the long-term autonomic evaluation in patients undergoing selective vagal denervation in the treatment of paroxysmal atrial fibrillation. They have evaluated the effects of vagal tone as a trigger to atrial fibrillation (AF) in a subset of patients. A new approach aiming selective ablation of the left atrium autonomic ganglia has been described. Conventional AF ablation leads to a transient autonomic system modification. Direct ablation of Ganglionated Plexi could lead to a more persistent modification. They have concluded the following: Autonomic modification persisted after long-term outcome when selective atrial vagal denervation guided by evoked vagal reflex was performed to treat paroxysmal AF. Another investigators team from Sao Paulo, Brazil (Drs. Pisani CF. et al.) had evaluated the feasibility of the autonomic heart plexus localization with epicardial and endocardial cardioneurostimulation. It is well known that the autonomic nervous system modifies eletrophysiologic properties of both atria, predisposing the appearance of atrial fibrillation (AF). Autonomic heart innervations are composed of parasympathetic ganglia and sympathetic post-ganglionar fibers over epicardial surface. They had concluded that parasympathetic ganglia mapping is possible to be done with cardio-neuron-stimulation. Many other teams as well such as Dr. Mauricio Arruda team (Combined Spectral Mapping Guided AF-Nests Ablation and Pulmonary Vein Antrum Isolation: A New Approach to Improve AF Ablation Success, Sao Paulo, Brazil. They have used the real time spectral mapping (SM) in sinus rhythm (SR) identifies sites with fibrillar atrial myocardium with high and disorganized frequencies [resonant], namely AF-Nest (AFN). Ablation of AFNs in both atria has been successfully used as an alternative approach to pulmonary vein isolation (PVI) in pts with atrial fibrillation (AF). PVI, at its antrum level (PVAI), combined with isolation of the superior vena cava (SVCI) have decreased post procedure (>2 months) AF recurrences to 17% in paroxysmal (PAF) and 30% in persistent/permanent AF. The potential benefit of AFN ablation added to PVAI was evaluated in this prospective, randomized study. They have concluded that AFN ablation guided by real-time SM in SR can safely be performed without significant additional procedure time and radiation exposure. The decrease in AF recurrence suggests AFN ablation may be considered as an adjunct to PVAI.
10) A team of investigators from Munich, Germany (Drs Deisenhofer I., Zrenner B. et al.,) had issued a randomized study (98 patients) on the effects of additional ablation of complex fractionated atrial electrograms. They have reported that segmental pulmonary vein isolation (PVI) cures paroxysmal atrial fibrillation (AF) in 65-75% of patients (pts). Ablation of complex fractionated atrial electrograms (CFAE) has emerged as an alternative ablation approach. This prospective randomized study evaluates the additional effect of CFAE ablation in pts with paroxysmal AF after PVI. They have concluded that “In paroxysmal AF, combination of PVI with ablation of CFAE showed in the intention-to-treat analysis no advantage to PVI alone (73% vs. 75% in sinus rhythm). However, patients without AF inducibility after PVI alone and pts undergoing additional CFAE ablation due to inducible AF showed a trend towards a better rhythm outcome”.
11) A team of investigators from the Catholic University, Rome, Italy (Elayi C., et al.; Pappone C. et al.,) in collaboration with two others local teams (Saliba W. Natale A. Arruda M. et al., Ontario, Canada and Cleveland, Ohio) had compared the results of three common ablation strategies for permanent atrial fibrillation. They have used 144 consecutive patients with permanent atrial fibrillation for a first catheter ablation procedure were randomized in three groups: 1) Anatomic circumferential PV ablation; 2) Pulmonary vein antrum isolation (PVAI) and 3) Hybrid approach (Ablation of complex fractionated atrial electrograms followed by PVAI). Success of the procedure was defined as the absence of atrial fibrillation occurring beyond two months after the procedure with patients off antiarrhythmic medication. Patients that had recurrence of atrial fibrillation were encouraged to have a second ablation. They had concluded that their study demonstrated the hybrid approach (CFAE and PVAI) had the best outcome after a single procedure. The CPVA was significantly less effective to maintain sinus rhythm than the two other strategies after one or two procedures.
12) A team of investigators from Taipei, Taiwan (Drs. Lin Y J. et al.,) have used the spectral analysis and frequency mapping in order to predict the abnormal atrial substrate in atrial fibrillation episodes. They have concluded that spectral analysis during sinus rhythm and coronary sinus pacing can detect abnormal atrial substrate that is critical for the persistence of atrial fibrillation. Therefore, known the locations of the highest dominant frequency may provide a useful target for the use of catheter ablation for the treatment of atrial fibrillation.
13) A team of investigators from Lisbon, Portugal (Drs. Oliveira M. et al.,) had evaluated the modifications of the autonomic activities during head up tilt testing in patients with paroxysmal atrial fibrillation based on the analysis of wavelet. Autonomic nervous system (ANS) plays a role in paroxysmal atrial fibrillation (PAF). Changes in autonomic control of heart rate variability (HRV) occur during orthostatic stress. Fourier Transform analysis cannot be applied to short transient phenomena related to ANS activity. The aim of their study was to evaluate, with wavelet analysis, HRV in 21 PAF patients and healthy subjects during head up tilt test. They have concluded that patients with PAF have a decreased low frequency and high frequency power during tilting movement without significant variations in the first minutes of head up tilt test; furthermore, the patients have decreased low frequency/high frequency values during the 2nd min of the tilt table procedure. Therefore, they had suggested that wavelets analysis might provide new insight into the assessment of autonomic heart regulation and underscore the presence of ANS abnormalities in PAF patients.
14) Many teams of investigators in the North America (Drs. Packer D., Natale A., Saliba W., Dibs S., Guiraudon G M, Scherlag B, Lazzara R., Jackman W., Lemery R., Nattel S., Armour A., etc.) have followed the claims as published in the U.S. Pat. No. 6,511,500 for the treatment of atrial fibrillation. They have intensively using this discovery in many fields such as “Clinical Electrophysiology and Pharmacology, Electrophysiology, Neurology, and Anatomy in the treatment of atrial fibrillation”. For example, Dr. Lemery had presented this topic as disclosed in the U.S. Pat. No. 6,511,500 patent: “Significant vagal denervation occurs in patients undergoing LA circumferential catheter ablation of atrial fibrillation”. Ganglionated plexuses can be precisely localized by using the high frequency stimulation. The locations of the ganglion plexuses were correlated with presence of complex fractionated atrial electrograms during atrial fibrillation. A vagal response during atrial fibrillation ablation was occurred infrequently. However, objective documentation of vagal denervation could be demonstrated acutely in 85% of previously positive sites. Drs. Scherlag B J., Lin J., Zhou J., Song J., Zhang Y., Lazzara R., Patterson E., Po SS and Jackman W M have clearly stated that their fundamental study provides functional evidence for the interconnections between ganglionated plexi to modulate sinus and AV nodal function, supporting clinical evidence that interconnections within the intrinsic cardiac ANS are critical elements in identifying the targets for atrial fibrillation ablation. Drs. Natale A team from Cleveland Clinic Foundation (Mauricio Arruda, MD, Jose Carlos Pachon, MD, Claude S Elayi, MD, Marketa Kozelulova, MD, Andrea Conrrado, MD, Tamer S Fahmy, MD, J. David Burkhardt, MD, Robert A Schweikert, MD, Walid Saliba, MD, Henrique Pachon, MD, Andrea Natale, MD, FRCP) have investigated hybrid strategies of ablating methods for permanent atrial fibrillation as an adjunct to pulmonary vein isolation. They have compared atrial fibrillation ‘nest’ in sinus rhythm to complex fractionated atrial electrograms. The recurrence of atrial fibrillation and atrial tachycardia after catheter ablation was monitored during at least 6 months on 163 patients. They have concluded that ablation of AFN or CFAE in adjunct to PVAI equally decreases total recurrences. However, the AFN ablation is more localized and requires shorter RF applications, thus preserving more myocardium, than CFAE ablation. AFN ablation is associated with lower AF recurrences, suggestive of a more specific substrate modifying strategy.
15) Dr. Shiroshita-Takeshita (Tokyo, Japan) and Dr. Hong Euy Lim (from the Lab of Dr. Young-Hoon Kim) “Driving mechanisms of acutely onset of vagal atrial fibrillation under the vagal stimulation”. Dr. Yong-Hoon Kim had revealed a new third fat pad nearby the IVC-left atrium and aorta in a canine open heart model; the effective refractory periods and atrial fibrillation inducibility were determined (5 V, 2 ms, 20 Hz). A non-contact mapping system (ESI 3000) has been used to analyze the propagating wavefronts by potential maps and dominant frequency of virtual ECGs obtained by Fast Fournier Transform (FFT) during AF on/off—vagal nerve stimulation; exactly as I had previously described in the patent U.S. Pat. No. 6,511,500 from the 1995's results. The results were also supported to the said '500 patent by confirming that vagal stimulation: 1) shortened atrial effective refractory periods at all sites, 2) increased the inducibility of atrial fibrillation (easily by premature extrastimuli), 3) increased the maintenance of atrial fibrillation (it was maintained as long as VNS was continued), 4) initiated/generated rapid reentrant wavelets with similar propagating route repetitively identified at the area between left atrium and left pulmonary veins. The OFF—VNS had converted AF to the flutter—like activations followed by spontaneous termination. Another team of investigator (Drs. Furukawa T., Hirao K, et al.,; from Department of Cardiovascular Medicine, Tokyo, Japan) had investigated the different effects of the autonomic tone on the occurrence of pulmonary vein origin atrial fibrillation in normal heart dogs and chronic atrioventricular block. It has been demonstrated that autonomic nerve stimulation is very important for the occurrence and maintenance of atrial fibrillation (AF) originating from pulmonary veins in normal heart dogs. However, there are no reports on the effects of autonomic tone on AF originating from the pulmonary veins in chronic heart failure dogs. The aim of the study was to compare the autonomic stimulation effects on PV origin AF in the dogs with normal and failed hearts. In conclusions, these findings had suggested that autonomic stimulation as a promoter of PV origin AF differs with the condition of the heart; VS promotes AF in normal hearts, but ISO promotes AF in failed heart.
16) A team from Helsinki University Hospital, Helsinki, Finland (Drs Heikki Väänänen, Jouni Kuusisto, Hannu Parikka, Markku Mäkijärvi, Lauri Toivonen, Räija Koskinen) had studied this topics. They have concluded that “Paroxysmal lone AF does not lead to atrial electrical of mechanical remodeling even over prolonged time, particularly the focal type of AF. These observations favor the concept that lone atrial fibrillation can be cured by successful elimination of the local arrhythmia substrate”.
17) Drs Bettoni M, Zimmermann M, from Geneva, Switzerland have studied the mechanisms favoring the occurrence of paroxysmal atrial fibrillation such as analyzed dynamic changes in autonomic tone preceding the onset of atrial fibrillation. They have concluded that the occurrence of paroxysmal atrial fibrillation depended on the variations of the autonomic nervous system tone. It had a primary increase in sympathetic tone followed by an abrupt shift toward parasympathetic dominance tone.

### C. Statistics Data and Analysis for the Invention

The currents methods and technologies for ablation of atrial fibrillation were combined for statistical analysis. The data were focused on early and late recurrence episodes of atrial arrhythmias after multiple and regional catheter ablation procedures used during the treatment of permanent atrial fibrillation.

### 1—Recurrence of Atrial Arrhythmias Following a Catheter Ablation Procedure Targeting the Right Atrium:

33% (Atrial Flutter; 21±15 months; study published in 2003).
10% (Atrial Flutter; 16±2 months; study published in 2002).
38% (Atrial Flutter; 28 months; study published in 2002).
39% (Paroxysmal atrial fibrillation; 22±11 months; study published in 2000).
29% (Atrial Flutter; 21±11 months; study published in 2000).
29% (Type: Atrial Flutter), 47% (Atrial Flutter and paroxysmal Atrial Fibrillation; Duration: 8±3 months; study published in 2000).
   2—Recurrence of atrial arrhythmias following a catheter ablation procedure targeting the left atrium:
37.2% (Type: Atrial Tachycardia; 23.7 months; study published in 2008)
36% vs. 55% (paroxysmal vs. persistent AF) (Type: 46% recurrence of Atrial Fibrillation; 12.7 months; study published in 2008).
47% (Type: Persistent AF; 18-24 months, ≈20 months; study published in 2007)
Three different methods in the left atrium: (38 with AF+7 with AT=45%; 11 with AF+25 with AT=36%; 22 with AF+14 with AT=36%; Duration of 11 months; study published in 2006).
63% (Symptomatic Atrial flutter; 12 months; study published in 2005).
49% (Type: Paroxysmal Atrial fibrillation; Duration: 12±5 months; study published in 2004).
Others.

### 3—Recurrence of Atrial Arrhythmias Following a Catheter Ablation Procedure Targeting the Right and Left Atrium:

Early recurrence rate was 31% (at 12 months). Late recurrence rate for permanent AF was 20% (at 24 months; study published in 2007).
24% (Permanent Atrial Fibrillation; 8.4 months; study published in 2006).
48% (38% Paroxysmal and 55% non-paroxysmal Atrial Fibrillation; 26±11 months; study published in 2006).
30% (Persistent Atrial fibrillation; 12 months; study published in 2006).
0% (13±7.4 months follow-up, study published in 2006).
Recurrence of permanent AF was 30% at >2 months (early recurrence would be estimated from 3 months) (study published in 2006).
50% recurrence with AF at a follow-up of 22±9, 8 months (study published in the Journal of the American College of Cardiology, 2006).
38% (Type: Atrial Flutter; Duration: 28 months; study published in 2002).

### 4—Recurrence of Atrial Arrhythmias Following a Catheter Ablation Procedure Targeting the Right Atrium in the Presence of Anti-Arrhythmic Drugs:

27% (Atrial flutter; 16±13 months; study published in 2003).
40% (Type: Atrial Flutter and Paroxysmal Atrial Fibrillation; Duration: 8±3 months; study published in 2000).
53% (Atrial flutter; 21±11 months; study published in 2000).

### 5—Recurrence of Atrial Arrhythmias Following a Catheter Ablation Procedure Targeting the Left Atrium in the Presence of Anti-Arrhythmic Drugs:

Recurrence rate of AF was 13.8% during 6.2±0.8 months (study published in 2007).
25%, 37% and 25% (Paroxysmal, Persistent and Permanent Atrial Fibrillation, 13±1 months; study published in 2006).
68% (Paroxysmal Atrial Fibrillation; 21±11 months; study published in 2006).
63% (Symptomatic Atrial Fibrillation; 12 months, study published in 2005).
37% (Type: Paroxysmal Atrial Fibrillation; Duration: 12±5 months; study published in 2004).

### 6—Recurrence of Atrial Arrhythmias Following a Catheter Ablation Procedure Targeting the Left Atrium and Right Atrium in the Presence of the Anti-Arrhythmic Drugs:

36% AF Recurrence for duration of 8.3±2 months (Type of recurrence: AT or AF; study published in 2007).
30% (Persistent Atrial Fibrillation; 12 months; study published in 2006).
34% (Type: Paroxysmal Atrial Fibrillation; Duration: 30±11 months; study published in 2003).
   7—

TABLE
The rate of recurrence of atrial arrhythmias (%) following the
treatment of atrial fibrillation (catheter ablation and drugs) with follow-up duration
(months):

| Ablation Targets | | | | | | |
|---|---|---|---|---|---|---|
| (/Methodology) | RA | LA | RA + LA | RA + AA | LA + AA | RA + LA + AA |
| Rate of Recurrence | 30 ± 11 | 35 ± 16 | 30 ± 15 | 40 ± 13 | 38 ± 20 | 33 ± 3 |
| Mean ± SD (%) | | | | | | |
| Duration Follow-up | 19 ± 7 | 14 ± 7 | 16 ± 9 | 15 ± 7 | 13 ± 5 | 17 ± 12 |
| Mean ± SD (months) | | | | | | |
| Number (Studies) | 6 | 30 | 9 | 3 | 7 | 3 |
| Statistically | NS | NS | NS | NS | NS | NS |
| Significance | | | | | | |

NS: Statistically not significance
RA: Right atrium
LA: Left atrium

8—Statistics Analysis for the Recurrence of Atrial Arrhythmias Followed by the Treatment of Atrial Fibrillation based on the Catheter Ablating’ Sites:

### Sunday, Apr. 6, 2008, 18:52:14

### One Way Analysis of Variance

### Normality Test Passed (P=0.3612)

### Equal Variance Test: Passed (P=0.1201)

| Group | N | Missing |
|---|---|---|
| RA − Abl | 6 | 0 |
| LA − Abl | 30 | 0 |
| (RA + LA) − Abl | 9 | 0 |
| RA − Abl + AA | 3 | 0 |
| LA − Abl + AA | 7 | 0 |
| (RA + LA) − Abl + AA | 3 | 0 |

| Group | Mean | Std Dev | SEM |
|---|---|---|---|
| RA − Abl | 29.7 | 10.54 | 4.30 |
| LA − Abl | 35.0 | 16.46 | 3.00 |
| (RA + LA) − Abl | 30.1 | 15.10 | 5.03 |
| RA − Abl + AA | 40.0 | 13.00 | 7.51 |
| LA − Abl + AA | 38.4 | 20.16 | 7.62 |
| (RA + LA) − Abl + AA | 33.3 | 3.06 | 1.76 |

Power of performed test with alpha = 0.0500: 0.0496

The power of the performed test (0.0496) is below the desired power of 0.8000. You should interpret the negative findings cautiously.

| Source of Variance | DF | SS | MS |
|---|---|---|---|
| Between Treatments | 5 | 519.6 | 103.9 |
| Residual | 52 | 13031.6 | 250.6 |
| Total | 57 | 13551.1 | |

| Source of Variance | F | P |
|---|---|---|
| Between Treatments | 0.415 | 0.8365 |
| Residual | | |
| Total | | |

The differences in the mean values among the treatment groups are not great enough to exclude the possibility that the difference is due to random sampling variability; there is not a statistically significant difference (P=0.836).

### 9—Statistics Analysis for the Duration of the Follow-up:

### Sunday, Apr. 6, 2008, 19:00:10

### One Way Analysis of Variance

### Normality Test Failed (P=<0.0001)

Test execution ended by user request, ANOVA on Ranks begun

### Sunday, Apr. 6, 2008, 19:00:10

### Kiruskal-Wallis One Way Analysis of Variance on Ranks

| Group | N | Missing |
|---|---|---|
| RA − FU | 6 | 0 |
| LA − FU | 30 | 0 |
| (RA + LA) − Abl) − FU | 9 | 0 |
| RA − Abl + AA − FU | 3 | 0 |
| (LA − Abl + AA) − FU | 7 | 0 |
| RA + LA)Abl + AA − FU | 3 | 0 |

| Group | Median | 25% | 75% |
|---|---|---|---|
| RA − FU | 21.0 | 16.00 | 22.0 |
| LA − FU | 12.0 | 11.00 | 12.0 |
| (RA + LA) − Abl) − FU | 13.0 | 11.00 | 24.5 |
| RA − Abl + AA − FU | 16.0 | 10.00 | 19.8 |
| (LA − Abl + AA) − FU | 13.0 | 12.25 | 15.3 |
| RA + LA)Abl + AA − FU | 12.0 | 9.00 | 25.5 |

H = 5.12 with 5 degrees of freedom.
(P = 0.4012)

The differences in the median values among the treatment groups are not great enough to exclude the possibility that the difference is due to random sampling variability; there is not a statistically significant difference (P=0.401).

### 10-Major Implications of the Autonomic Nervous System on Atrial Arrhythmias:

a) Anatomy concepts: Autonomic heart innervations are composed of parasympathetic ganglia and sympathetic post-ganglionar fibers over epicardial surface. Recent basic study (Pauza D. et al.,) has revealed that extracardiac nerves from the mediastinum go into the subepicardium through the specific site bounded by the serous pericardium (around the great heart veins on the heart base; and around the pulmonary trunk and ascending aorta). Several basic research studies have showed a complex and interconnected nerve structures system in cardiac chambers (Pauza, D. et al., Armour A. et al., Coumel, P.). However, the shapes of those nerve structures and their anatomical locations within a cardiac chamber have species-dependent properties. Thus, may limit the use of anatomical data from other species to determine the exact anatomical’ locations of specific sites for local treatment of clinical cardiac arrhythmias, such as for the use of catheter ablation and/or for the use of local infusion of autonomic nervous system’ antagonists. Nevertheless, the electrophysiological proprieties found on the basic studies are useful for the clinical developments and benefits.

Furthermore, the epicardial neural plexus in humans studies (Pauza et al.,), by using staining for acetylcholinesterase, provides a morphological framework for an understanding of how intrinsic ganglia and nerves are structurally organized within the human heart (methodology: morphology of the intrinsic neural plexus was revealed by a histochemical method for acetylcholinesterase in whole hearts and examined by stereoscopic, contact, and electron microscopes).

b) Electrophysiology concepts: Autonomic nervous system modifies electrophysiological properties of both atria, predisposing the appearance of atrial fibrillation. Dominant frequency mapping during persistent atrial fibrillation identifies high-frequency atrial sites. Dr. Dibs S et al., hypothesizes that high-frequency atrial sites represent AF drivers, and as such would be expected to show not only higher frequency, but also higher regularity and centrifugal decrease in frequency. Dr. Dibs results revealed that high-frequency sites appeared in the left atrium during persistent episodes of atrial fibrillation, manifest higher regularity and centrifugal dissipation of frequency. Thus suggest that high-frequency sites act as drivers for persistent episodes of atrial fibrillation.

Dr. Hou Y et al., has investigated the functional interactions between ganglionated plexi within the intrinsic cardiac autonomic nervous system, his study had provided functional evidence for the interconnections between ganglionated plexi to modulate sinus and atrio-ventricular nodal function, supporting clinical evidence that interconnections within the intrinsic cardiac of the autonomic nervous system are critical elements in identifying the targets for atrial fibrillation ablation. Dr. Brooks A et al., had demonstrated that Complex fractionated electrograms and highest dominant frequency were potential targets for the ablative treatment of atrial fibrillation. The model used was A NavX system which utilizes point-by-point electrogram acquisition and analysis to project complex fractionated electrogram or dominant frequency data transformations on 3D maps. He had concluded that a global atrial estimates of complex fractionated electrogram and dominant frequency correlate inversely between patients. However, in persistent and permanent atrial fibrillation there was no significant relationship between those two parameters for individual points which could suggests that those two electrophysiological parameters (complex fractionated electrogram and dominant frequency) maps might provide independent diagnostic information during atrial fibrillation episodes. Dr. Lu Z et al., has found that dominant frequency and complex fractionated electrograms may be caused by enhanced activity of the intrinsic cardiac autonomic nervous system. They have ACH applied acetylcholine at right atrial sites which induced rotor as electrograms with the highest dominant frequency; the sites closest to right atrial appendage have showed the lowest prevalence of complex fractionated electrograms compared to those closest to the Ganglionated Plexi. They have suggested that complex fractionated electrograms might result from activation of neural activity from the right atrial appendage rotors rather than fibrillary from the myocardial conduction. Therefore, the ablation of the ganglionated nerve had eliminated rotor and complex fractionated electrograms and decreased dominant frequencies at all sites. Dr. Scherlag B J. Et al., had concluded according to his basic study on 12 dogs that autonomic Ganglionated Plexi stimulation, subthreshold for atrial excitation, converts isolated premature depolarization into atrial fibrillation, suggesting that autonomic tone may play a critical role in the initiation of paroxysmal atrial fibrillation.

c) Mechanisms of Autonomic Nervous System Effects and Behind:

The effects of sympathetic neural activity on the heart are gradually developed and receded, whereas the inhibitory effects of vagal activity appear and disappear rapidly. The automatic cells in the heart respond promptly to vagal stimulation within a steady-state value of two cardiac cycles. The ability of the vagus nerves to regulate heart rate beat by beat could be explained by the speed at which the neural signal is rapidly transduced to a cardiac response and by also by the rapidity of the processes that restore the basal heart rate when vagal activity ceases. The mechanisms of this rapid development of vagal effects on heart rate will be related to: 1) the acetylcholine regulated potassium channels; 2) the hyperpolarization activated channels, which conduct the If current; and 3) the calcium channels. The acetylcholine and the If channels could both respond rapidly to vagal activity. The If and Ica channels are directly involved in generating the slow diastolic depolarisation in sinus node cells. The release of acetylcholine interacts with cardiac muscarinic receptors that are coupled to its regulated potassium channels directly through G proteins without an interaction of a slow second messenger system. These potassium channels are fully activated by this release of acetylcholine within a few milliseconds. The relatively slow development of the sympathetic responses has been attributed mainly to the inclusion of a second messenger system, notably the adenylyl cyclase system, in the cascade of events that transduce the neuronal release of norepinephrine into a change in cardiac performance. The chronotropic response of the heart to sympathetic activation is mediated mainly via several types of ion channels, such as Ica and If currents. This second messenger system is too slow to permit beat-by-beat regulation of cardiac function. The norepinephrine released from the sympathetic nerve endings is removed from the cardiac tissues much more slowly than is the acetylcholine that is released from the vagal terminals. Then, the atrial tissue and the related ionic currents (Ica, If) are submitted to the sympathetic neural activity after a certain delay of ganglion stellar stimulation. Furthermore, during the study of vagal-sympathetic interaction, the on-set effects of sympathetic stimulation are considered in the presence of existing vagal stimulation effects on atrial tissue and not in the on-set of this vagal stimulation effects.

d) Methods Based on Anatomical-Electrophysiological Concepts:

Autonomic nervous system activation decreases the effective refractory period and the conduction velocity; therefore, it increases the duration of the P wave. A new approach to monitor the successful and complete attenuation of the autonomic nervous system would be confirmed by a significant decrease of the P wave's durations (75%). It has been reported that reverse atrial remodeling after PVI is demonstrated by a shortening of the P wave duration on the 12 lead ECG. Therefore, a decrease in P wave duration is predictive of a successful GP ablation. Dr. Bauer A. et al., has described the degree of long term efficacy of a method correlated to the vagal. Two methods were described: a Circumferential and segmental pulmonary vein ablation were two established interventional treatment strategies for ablation of atrial fibrillation. Both techniques require the application of radiofrequency current at anatomical sites that are close to parasympathetic ganglia. Vagal activity is known to be strongly involved in the triggering and maintenance of atrial fibrillation. Both methods were induced an immediate decrease of vagal tone. However, while this decrease is only transient with segmental, it persists with circumferential pulmonary vein ablation for at least one year. Dr. Prasad SK. et al., has reported that intrinsic autonomic activity within the smooth muscle sleeves of the pulmonary veins can trigger electrical activation which leads to atrial fibrillation. In hypothesized that pulmonary vein ostia area, as determined by multi-detector computed tomography, would predict recurrent of atrial fibrillation after pulmonary vein antral isolation; he concluded that a smaller total pulmonary vein ostia area (with intrinsic autonomic activity), particularly of the left sided of the pulmonary veins, as measured by multi-detector computed tomography was associated with recurrent atrial fibrillation in patients undergoing pulmonary vein antral isolation. Electron microscopic of intrinsic cardiac ganglia in the adult human study: Cardiac ganglion cells in healthy persons appear to be directly involved in the development of atrial and ventricular arrhythmias, atrio-ventricular block, abnormal QT interval prolongation and sudden cardiac death. The results of Dr. Pauza et al., could be targeted for the development of new catheter ablation approaches for the treatment of cardiac arrhythmias and in particular for the treatment of atrial fibrillation. These results of their preliminary study provide baseline information on the normal ultra structure of intra-cardiac ganglia in healthy humans which may be useful for assessing and interpreting the degree of damage of ganglion cells in autonomic neuropathies of human heart.

Drs. Jackman, W M., Lazzara R., Patterson E., Scherlag B J et al., have studied the functional interactions between ganglionated plexi within the intrinsic cardiac autonomic nervous system. They have concluded that a functional evidence for the interconnections between ganglionated plexi to modulate sinus and AV nodal function, supporting clinical evidence that interconnections within the intrinsic cardiac autonomic nervous system are critical elements in identifying the targets for atrial fibrillation treatment including the method of local ablation. Furthermore, several clinical investigators, including Dr. Warren Jackman have published and confirmed the direct and distinctive relationship or requirement for some degree of stimulation from the Ganglionated Plexi in order to have Complex Fractionated Electrograms. Therefore, any tools, software, program, device, catheter, etc, that contain in their indication the identification of Complex Fractionated Electrogram or the dominant frequency, are entitled to identify the Ganglionated Plexi location. In consequence, it could be used for the treatment of atrial fibrillation as described and covered by the U.S. Pat. No. 6,511,500 patent claims. A study was con ducted by Dr. Roux J F was demonstrated a significant correlation between two complex fractionated electrograms maps constructed 32 minutes apart with 80% concordance of complex fractionated electrogram sites. This likely represents stability of the underlying atrial substrate and/or “drivers” of atrial fibrillation. Drs. Lu Z., Scherlag B J., Po S. and Jackman W M study on the autonomic nervous basis for differential dominant frequency and complex fractionated electrograms during the occurrence of atrial fibrillation. They have concluded that acetylcholine applied at right atrial appendage induced rotor-like electrograms with the highest dominant frequency and the lowest prevalence of complex fractionated electrograms compared to those closest to the ganglia. Their results suggested that complex fractionated electrograms may result from activation of neural conduction from rotors rather than fibrillary's myocardial conduction. Therefore, the ablation of ganglionated plexi eliminated rotor and complex fractionated electrograms and decreased dominant frequency at all sites.

Repeat ablation procedure guided by the complex fractionated electrograms for targeting the autonomic nervous structure in the myocardium for chronic atrial fibrillation treatment had demonstrated a higher efficacy (>40%; Oral H., Morady F. et al.,) A team from the University of Virginia, Charlottesville, Va., USA (Drs. Subramanian R, Huffman M, Prudente L et al.) have recently investigated the recurrence of atrial fibrillation on 210 consecutive patients who had pulmonary vein isolation (109 patients have received additional linear lesions and/or ablation of complex fractionated atrial electrograms, 54 patients had more than one ablation). The follow-up data was obtained by chart review and scripted interviews. The recurrence after the last ablation was defined as documented AF or AFL, or prolonged palpitations that the patient attributed to AF. The number of patients with recurrent AF was assessed at each of the following time-points: 0-1, 1-3, 3-6, 6-12, 12-18, 18-24, 24-36 months. The results were for the recurrence was as the following:
For persistent and paroxysmal AF, the recurrence rate was 40% for a period of 20±7 months.
For persistent AF, the recurrence rate was 11% at 3-12 months; 44 at 12-24 months
The Figure demonstrates incidence of recurrence over each time period (0-1, 1-3, 3-6, 6-12, 12-18, 18-24, 24-36 months). At the 18-24 months time period, the rate of recurrence in PAF pts plateaus at 19%. In contrast, there was a steady rise in the rate of recurrence in persistent AF that achieves 47% at 18-24 months (p=0.02). The presence of structural heart disease, age, gender, and the lesion set were not predictive of recurrence. The authors have concluded that amongst patients with paroxysmal AF, freedom from recurrence at 6-12 months after ablation suggests a good long term result. However, in persistent AF late recurrence is common (a correlation between the incidence of recurrence and the time-periods, specifically for persistent of AF).

Drs. Packer team from Mayo Clinic, MN have disclosed as well the increasing rate of the recurrence over time (beyond 18 months) and more specifically for the persistent atrial fibrillation. Furthermore, they were able to questioning the concept of recurrence in persistent AF as a result from an additional substrate-mediated arrhythmia which was not be modifiable by the catheter ablation manner.

This invention is demonstrating and clarifying for the first time that those additional substrates would be related to the autonomic nervous system regenerative substrates such activities/tonicities/anatomies/restructuring/etc (re-activation, re-establishment, ionic and/or molecular changes, etc.). Therefore, a correlation between the recurrence of AF rate and the time-period would support my scientific interpretations and observations on the primordial role of the autonomic nerve regenerative (or re-establishment) for the incidence of recurrence of atrial arrhythmias following the treatment of atrial fibrillation over time (specifically following the treatment of the persistent ones).

1: A method of treating the recurrence of atrial arrhythmias, wherein delivering energy via catheter ablation ^{π} to specific areas of the epicardium, the method comprising the step of:
Inserting an electrophysiology ablation catheter having a tip section with an ablation electrode pointed toward the right or left atrial epicardium and directing the catheter to specific autonomic nerve structure stabilizing the ablation electrode at said epicardial nerve structure; and
delivering effective energy through the electrode sufficient to attenuate the electrophysio logical autonomic signals” generated by this area and causing the recurrence and perpetuation of the said atrial arrhythmias.

2. A method of treating the recurrence of atrial arrhythmias, wherein delivering energy via catheter ablation ^{π} to specific areas of the endomyocardium, the method comprising the step of:
Inserting an electrophysiology ablation catheter having a tip section with an ablation electrode into the right or left atrial chambers and pointed toward the endomyocardium and directing the catheter to specific autonomic nerve structure;
stabilizing the ablation electrode at said endocardial nerve structure; and
delivering effective energy through the electrode sufficient to attenuate the electrophysiological autonomic signals ^{Ω} generated by this area and causing the recurrence and perpetuation of the said atrial arrhythmias.

3. A method of treating the recurrence of atrial arrhythmias via denervating directly an autonomic nerve structure *, the method comprising the steps of:
using a multi array real time mapping system ^{δ} for identifying areas of autonomic nerve structures generated electrohysiological autonomic signals ^{Ω} which lead to the occurrence of atrial arrhythmias;
locally delivering an effective energy ^{λ} through the electrode sufficient to attenuate the electrophysiological autonomic signals generated by this area and causing the recurrence and perpetuation of the said atrial arrhythmias.

4. The method according to any one of claims 1, 2 or 3 further comprising the step of using additional tools ^{π}, such as a neurostimulator, in order to re-test the attenuation of the electrophysiological activation of an autonomic nerve structure ^{Ω}* thereby prevent the nerve structure from re-establishing innervations such as regenerative activities.

5. The method of claim 4, wherein said preventing autonomic nerve structure’ regenerative activities ^{Ω}* further comprises prevention of the recurrence of atrial arrhythmias.

6. The method of claim 4, wherein said preventing the endothelium from re-establishing innervations activities with an autonomic nerve structure” further comprises prevention of the recurrence of atrial arrhythmias.

7. The method according to any one of claims 1, 2 or 3 further comprising the step of locally infusing the endomyocardium by a vagal antagonist agent prevents the recurrence of atrial arrhythmias.

8. The method according to any one of claims 1, 2 or 3 further comprising the step of locally infusing the endomyocardium by vasoactive intestinal peptide compound treats the occurrence of atrial arrhythmias with an ability to prevent its recurrence.

9. The method according to any one of claims 1, 2 or 3 further comprising the step of locally infusing the endomyocardium by an ionic-channel of acetylcholine-sensitive K⁺—currents agent ^{Φ} prevents the recurrence of atrial arrhythmias.

10. The method according to any one of claims 1, 2 or 3 further comprising the step of locally infusing the epicardium by an ionic-channel of acetylcholine-sensitive K⁺−currents agent (prevents the recurrence of atrial arrhythmias.

11. The method according to any one of claims 1, 2 or 3 further comprising the step of infusing an anticoagulant agent prevents the occurrence of stroke.

12. A method of preventing the recurrence of atrial arrhythmias wherein administrating an I_{KACh} antagonist compound ^{Φ} to a patient treats and prevents the recurrence of atrial arrhythmias, the method comprising at least one of:
1) Oral administration of the said compound,
2) IV infusing the said compound,
3) a direct application of the compound via a drug eluting patch on the atrial epicardium, and
4) a direct application of the compound via a drug eluting catheter on the atrial endocardium. ^{λ}: A source of energy would included and not limited to radiofrequency stimulus, microwave stimulus, ultrasound stimulus and laser stimulus; cryoablation application would be included (even if it is not using or delivering a source of energy). ^{π}: comprising and not limited to ablation catheter(s), electrode(s), tools, surgical device(s), surgical tool(s) and/or tool(s) for neurostimulating for the epicardium, myocardium and/or endocardium. *: Autonomic nerve structure is defined as any autonomic nerve located on the cardiac myocardium, epicardium and/or endocardium; the nomination of such nerve structure could be technically modified and used in the literature for charming matter, however, all terms and definitions lead to the same nerve structure which include and not limited to the ganglionated plexi, ganglia, ansa, axon, somata, dendrites, fibers, gap junction, synapse, inter- or intra-connection between two different part of the said nerve structure. ^{Ω}: Including and not limited to ectopic extra beats, high frequency, dominant frequency, complex fractionated atrial electrograms, significant decreases of the P wave's durations, autonomic bradycardia or tachycardia intensities, degree of heart rate variability (such R-R variations), AV block modification, and/or initiating of arrhythmia(s). ^{δ}: A mapping system which utilizes point-by-point electrogram acquisition and analysis to project complex fractionated electrogram, dominant frequency, any other autonomic nervous system data transformations on 2 or 3D maps and/or an x-ray procedure that uses the help of a computer to produce a detailed picture of a cross section of the cardiac endothelium (epicardium, myocardium, epicardium). Φ: including and not limited to I_{KACh} and/or multiple muscarinic acetylcholine-receptor subtypes that could mediate kinetically and biophysically distinct K⁺−currents such as the 1—I_{KACh} (M2 receptor blocker or blocker of I_{KM2}); 2—I_{KM3} (M3 receptor blocker) and 3—I_{KM4} (M4 receptor blocker).
